(19) United States
(12) Patent Application Publication
(10) Pub. No.: US 2007/0167837 A1
(43) Pub. Date: Jul. 19, 2007

(54) METHOD AND APPARATUS FOR THREE DIMENSIONAL SCANNING IN A VISIBLY LIT ENVIRONMENT USING STRUCTURED LIGHT

(75) Inventors: Eric P. Moyer, Huntington Beach, CA (US); Joseph R. Winston, Chapel Hill, NC (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: SEABOARD ENGINEERING CORP. (a New Jersey corporation), Mt. Laurel, NJ (US)

(21) Appl. No.: 11/564,974

(22) Filed: Nov. 30, 2006

Related U.S. Application Data
(60) Provisional application No. 60/748,041, filed on Dec. 7, 2005.

(51) Int. Cl. A61B 6/00; G01C 17/00; A61B 5/103

(52) U.S. Cl. 600/476; 702/152; 702/153; 600/587

(57) ABSTRACT

An apparatus for acquiring and processing data regarding physical attributes of a user in an accurate, repeatable and automated manner comprises a generally enclosed housing sized for receiving a user therein, the housing having an interior and an opening with a door for entry into and exit from the housing by the user, the door, when closed, preventing outside light from entering the housing. At least one structured light source is located within the housing for projecting structured light onto at least one portion of the body of a user to be scanned within the housing for obtaining spatial measurements of the at least one portion of the body of the user. At least one light detector is provided within the housing for detecting structured light reflected from the at least one portion of the body of the user and for generating electrical signals corresponding to the spatial measurements of the at least one portion of the body of the user. At least one source of controlled ambient light is located within the housing for emitting ambient light having controlled spectral components visible to the user

### CROSS-REFERENCE TO RELATED APPLICATIONS

This application claims the benefit of U.S. Provisional Patent Application No. 60/748,041 filed Dec. 7, 2005 and entitled “3D Scanning in a Visibly Lit Environment,” the entire subject matter of which is incorporated herein by reference.

### BACKGROUND OF THE INVENTION

The present invention relates to three dimensional (3D) scanning technology and, more particularly, to the implementation of a 3D scanner using structured light projected onto the scanned object where visible light is desirable in the scanning chamber or environment.

In the fields of health and fitness, there are a number of methods which are currently used for helping users determine their health/fitness state or status and progress in building muscle mass, losing weight and/or assessing overall health and fitness. The current methods include primarily manual measurement of the user's physical attributes such as height, weight, heart rate, body fat percentage and the size of waist, bust, abdomen, hips, thighs, calves, arms, etc. Convenience, accuracy and reliability are important to generate repeatable and accurate measurements in a manner that is acceptable to the user and provides an overall accurate assessment of health and fitness.

Such traditional approaches include the periodic taking of independent user measurements by manual means, including using tape measures, scales and other independent measuring devices. Typically, such measurements are taken by a health or fitness staff member and are recorded for purposes of tracking the state of the health/fitness of the user and the user's progress. The measurements often vary in accuracy and repeatability due to variations in the techniques employed by the various different staff members. Accuracy may also be compromised by a staff member in an effort to encourage, persuade or assist the user in attaining a stated performance goal. The ability of the staff member to affect the measurement process in differing ways often leads to subjective, and not objective, data collection. While any such traditional measurements may each in their own turn be reliable, accurate and convenient, they do not constitute solutions which can be said to be at the same time reliable, repeatable, accurate and convenient.

U.S. patent application Ser. No. 10/981,008 (hereinafter referred to as “the '008 application”) filed Nov. 4, 2004 (U.S. Patent Application Publication No. 2996/0154708A1) and entitled “Apparatus and Method for Acquiring and Processing Data Regarding Physical Attributes of a User,” the subject matter of which is hereby incorporated herein by reference, discloses a method and apparatus for obtaining multiple human body measurements in an automated manner using a system that integrates three dimensional imaging of the user's body with direct measurement of the weight and heart rate of the user. The body measurements are all stored and can be used by the user or others to track his or her current health state as well as the progress of his or her health and fitness or can be sent to a health or fitness provider for the creation of specific assessments and plans. The present invention comprises an improvement upon the scanning methods and apparatus disclosed in the '008 application.

In the field of 3D scanning, the location in a three dimensional coordinate frame of points on the surface of an object or a person being scanned (a “point cloud”) can best be determined by viewing structured light projected onto that surface or person. The minimization of non-structured or ambient light reduces reflected light from sources other than the structured light to allow the signal-to-noise ratio at the light detecting devices to be improved, providing enhanced contrast for the detecting devices. Furthermore, since non-signal return must typically be removed from the detected image to eliminate spurious points, a dark scanning environment with little or no ambient light reduces the complexity and quantity of computational filtering.

Traditional approaches include the creation of a scanning chamber or environment such as disclosed in the '008 application that is designed to minimize visible light. In the case of scanning humans or other living beings, the experience of being scanned in a dark environment may produce unpleasant symptoms of claustrophobia, anxiety and disorientation, and may create a disincentive to use the 3D scanner.

The present invention provides controlled ambient light in a scanning booth such as the one disclosed in the '008 application having spectral components which are limited to bands attenuated by a filter at each detecting device, and structured light for 3D scanning that contains frequencies that are passed by a filter at each detecting device. This allows the scanned object to be bathed in visible ambient light, while each detecting device is only capable of viewing the structured light employed for the scanning process.

### BRIEF SUMMARY OF THE INVENTION

Briefly stated, in one embodiment, the present invention comprises an apparatus for acquiring and processing data regarding physical attributes of a user in an accurate, repeatable and automated manner. The apparatus includes a generally enclosed housing sized for receiving a user therein, the housing having an interior and an opening with a door for entry into and exit from the housing by the user, the door, when closed, preventing outside light from entering the housing. At least one structured light source is provided within the housing for projecting structured light onto at least one portion of the body of a user to be scanned within the housing for obtaining spatial measurements of the at least one portion of the body of the user. At least one light detector is located within the housing for detecting structured light reflected from the at least one portion of the body of the user and for generating electrical signals corresponding to the spatial measurements of the at least one portion of the body of the user. At least one source of controlled ambient light is provided within the housing for emitting ambient light having controlled spectral components visible to the user.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a top plan schematic diagram showing a preferred embodiment of an apparatus for obtaining 3D scan data using structured light under controlled visible light conditions;

FIG. 2 is a schematic block diagram showing a preferred embodiment of a detecting device of the type shown in FIG. 1;

FIG. 3 is a schematic block diagram showing the principal subsystems of a preferred embodiment of a device of the type shown in FIG. 1 to project structured light for the purposes of illuminating the surface of a scanned object; and

FIG. 4 is a schematic block diagram showing a preferred embodiment of a device of the type shown in FIG. 1 to supply controlled visible ambient light in a 3D scanning environment.

### DETAILED DESCRIPTION OF THE INVENTION

Referring to the drawings wherein the same reference numerals are used to indicate the same components throughout the several figures there is shown a preferred embodiment of the present invention which will now be described in detail. The below described preferred embodiment is only one implementation of a 3D body scanner with controlled ambient light at the blue end of the visible spectrum, low-pass light detector filters that attenuate the blue ambient light, and red laser structured light within the pass band of an optical filter of the light detector. However, it should be clearly understood that the present invention is not limited to the below described embodiment.

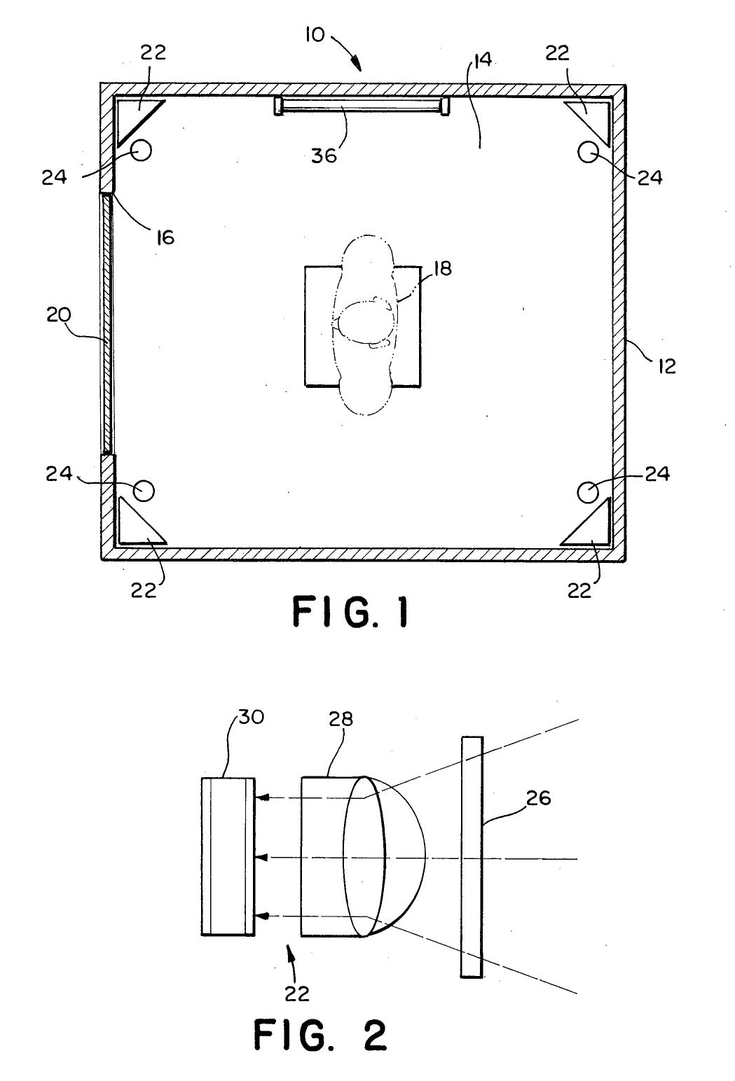

There is shown in FIG. 1 a top plan schematic diagram of a preferred embodiment of an apparatus 10 in accordance with the present invention for acquiring and processing data regarding physical attributes of a user 18. The apparatus 10 includes a housing 12, in the present embodiment a scanning booth, having a generally enclosed interior 14 and at least one opening 16 for entry into and exit from the scanning booth 12 by a user 18. The scanning booth 12 may be a generally permanent structure or may comprise a temporary, portable or mobile structure such as a tent, kiosk, truck or trailer mounted facility or the like. The scanning booth 12 may be located within a health club, YMCA, gymnasium or other facility or, if desired, may comprise a stand alone structure, for example, within a mall or shopping center, community center, club house or the like. Preferably, the opening 16 includes a tightly closing door 20 which may be opened and closed by the user 18 during entry to and exit from the housing 12. For reasons that will hereinafter become apparent, the door 20 is sized and shaped so that when closed, it effectively prevents undesired outside light from entering the interior 14 of the scanning booth 12, particularly through the area between the periphery of the door 20 and the opening 16. In some applications, weather stripping (not shown) or other such materials may be used to help block outside light from entering the scanning booth 12 through the area between the closed door 20 and the opening 16. In other applications, the door 20 may comprise a curtain, such as a blackout curtain, or a series of spaced curtains or the like.

In the present embodiment, the scanning booth 12 is generally rectangular shaped in plan view and is sized for receiving a user 18 therein. However, the scanning booth 12 could take on any one of a variety of different shapes or dimensions if desired. The purpose of the scanning booth 12 is to provide a facility to establish an enclosed space to provide privacy for the user 18 when measurements are being taken and to prevent outside light from entering the interior of the scanning booth 12, at least during the scanning process. It will be appreciated by the those of ordinary skill in the art that the size and shape of the scanning booth 12 as well as the location of the scanning booth 12 and the materials from which the scanning booth 12 may be built will vary from application to application and should not be considered to be a limitation on the present invention. For example, in one embodiment, the scanning booth 12 may comprise a tent or a series of curtains which define the enclosed interior 14 whereas in another embodiment, the scanning booth 12 may comprise actual walls formed of a material or combination of materials such as a polymeric material, wood, wood frame, steel or any other such suitable materials.

In the preferred embodiment, the scanning booth 12 contains at least one and preferably a plurality of light detector devices or light detectors 22 at spaced locations and at least one and preferably a plurality of structured light sources 24 at spaced locations within the scanning booth 12. In the embodiment shown in FIG. 1, there are four light detectors 22 and four structured light sources 24, one of each located proximate to each corner of the scanning booth 12. It should be understood that the number and locations of the light detectors 22 and/or the structured light sources 24 may vary depending upon the structure and size of the scanning booth 12 and depending upon the particular application. Each of the structured light sources 24 projects structured light onto at least one portion of the body of a user to be scanned within the scanning booth 12. Correspondingly, each of the light detectors 22 are for detecting structured light reflected from the at least one portion of the body of the user 18 and for generating electrical signals corresponding to the spatial measurements of the at least one portion of the body of the user 18. In this manner, a three dimensional point cloud of the surface of the user may be computed.

The scanning booth 12 further includes at least one source of controlled ambient light 36 for emitting ambient light having controlled spectral components which are visible to the user 18 but which are attenuated at the light detectors 22. In the preferred embodiment, the controlled ambient light source 36 is located on one wall of the scanning booth 12. However, the controlled ambient light source 36 could also be located at one or more other locations inside or outside of the scanning booth 12. A plurality of controlled ambient light sources 36 (not shown), preferably at spaced locations within the scanning booth 12 could be employed in particular applications.

FIG. 2 depicts a preferred light detector device or light detector 22. In the preferred embodiment, the light detector 22 includes one or more optical components to pass spectral components of the structured light reflected from portions of the body of the user 18 being scanned and to attenuate reflected or other spectral components of the controlled ambient light within the scanning booth 12. Preferably, the one or more optical components include at least one optical filter 26. In the present embodiment, the optical filter 26 is a low-pass filter that passes structured light having a frequency at or below the red end of the visible spectrum and attenuates light having a frequency above the red end of the visible spectrum and particularly light near the blue end of the spectrum. In the present embodiment, the optical filter 26 is fabricated of a polymeric material, in particular a polycarbonate. The optical filter 26, which could comprise more than one individual filter, could also be fabricated of other materials, and could be located separately from the rest of the light detector 22. Alternatively, the optical filter 26 could be implemented as one or more coatings (not shown). The optical filter 26 could be a high-pass filter, a low-pass filter, a band-pass filter or a band block filter, and could attenuate some or all of the visible light spectrum. For example, in an alternate embodiment (not shown), the optical filter 26 comprises a high pass filter that passes structured light having a frequency at or above the blue end of the visible spectrum and attenuates light having a frequency below the blue end of the visible spectrum and particularly light near the red end of the spectrum.

The light detector 22 further comprises an optical imager 30 for receiving light and converting the received light into electrical signals and an imager lens 28 for focusing light received from the filter 26 onto the optical imager 30. In the preferred embodiment, the imager lens 28 and optical imager 30 is comprised of a separate assembly which is attached to a printed circuit board (not shown). The imager lens 28 could be integrated into an assembly with the optical imager 30 or could be a separate component. The imager lens 28 is preferably of the convex/planer or some other type so that light passing through the imager lens 28 is focused on to the optical imager 30 as shown. In the preferred embodiment, the optical imager 30 is a VGA resolution black and white CMOS imager. Other optical imagers which may be used include but are not limited to variations in the size and aspect ratio of the optical imager 30 (e.g. SVGA, XGA, WXGA), quantity of imagers, imager type (CMOS, CCD), imagers that respond outside of the visible light spectrum (e.g. infrared) and color imagers. It should be understood that the type and/or construction of the optical imager 30 and/or the imager lens 28 should not be considered to be a limitation on the present invention.

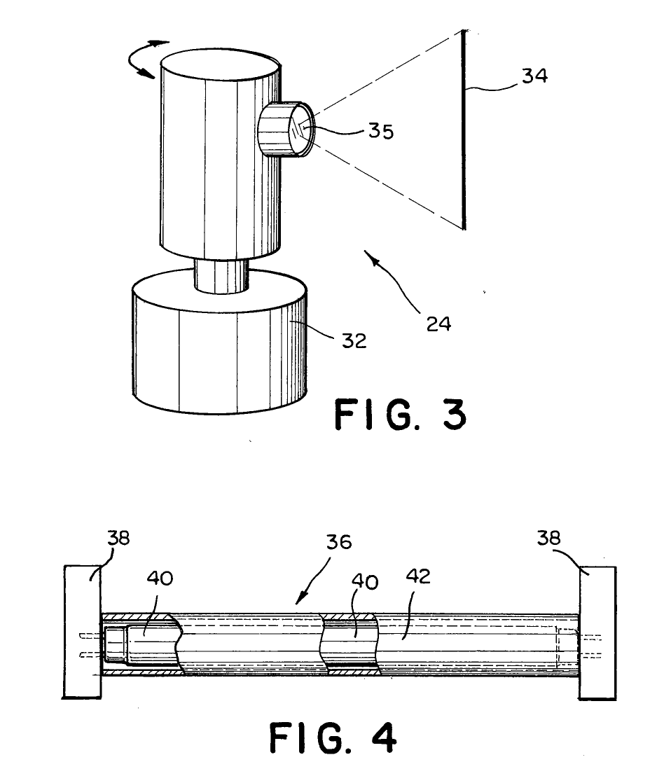

FIG. 3 depicts a structured light source 24 which projects structured light including the predetermined spectral components which are passed by the optical filter 26 of the light detector 22 described above onto the portion of the body of the user 18 being scanned. The structured light source 24 includes at least one of a broad spectrum light source, such as a fluorescent bulb, an incandescent bulb, a halogen bulb or the like or a narrow spectrum light source such as a laser, laser diode, light emitting diode or the like which is preferably within the band pass of the filter 26 of the light detector 22. The structured light source 24 could project light having a frequency which is within or outside of the visible spectrum if desired. In the preferred embodiment, the structured light source 24 includes a red 670 nm laser and lens assembly 35 which projects a red laser line 34 onto the portion of the body of the user 18 to be scanned, and is moved between incrementally located positions (e.g. side to side) by a stepper motor 32 of a type well known in the art. In other embodiments, the structured light source 24 could project a single point, many points, a single line, many lines, a pattern or patterns, a gradient or diffraction image. The structured light pattern may be stationary, may move, or may change over time.

FIG. 4 depicts a preferred controlled ambient lighting device 36. The ambient lighting device 36 emits visible light having controlled spectral components inside the scanning booth 12 depicted in FIG. 1. A plurality of controlled ambient light sources 36 could be positioned at spaced locations within the scanning booth 12 if desired. The controlled ambient light emitted by the ambient lighting device 36 has controlled spectral components such that some or all of the light components are attenuated by the filter 26 as shown in FIG. 2. In the preferred embodiment, the controlled ambient light is generated by one or more fluorescent light bulbs 40, each wrapped in an optical filter 42 that only passes light at the blue end of the spectrum and attenuates light below the blue end of the spectrum. The controlled ambient light source 36 is supported within the scanner booth 12 by a pair of support members 38, one on each end. The controlled ambient light source 36 could also be implemented with other types of light sources (e.g. incandescent, LED, halogen) with or without a filter 42 as required. The controlled ambient light source 36 could also use other types of filters or no filter at all, and could otherwise locate the filter (e.g. surrounding multiple light sources, or integrated into the light housing). By using controlled ambient light in this manner all or portions of the body of a user 18 may be successfully scanned while the user enjoys at least some visible light.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. An apparatus for acquiring and processing data regarding physical attributes of a user in an accurate, repeatable and automated manner comprising:
a generally enclosed housing sized for receiving a user therein, the housing having an interior and an opening with a door for entry into and exit from the housing by the user, the door, when closed, preventing outside light from entering the housing;
at least one structured light source within the housing for projecting structured light onto at least one portion of the body of a user to be scanned within the housing for obtaining spatial measurements of the at least one portion of the body of the user;
at least one light detector within the housing for detecting structured light reflected from the at least one portion of the body of the user and for generating electrical signals corresponding to the spatial measurements of the at least one portion of the body of the user; and
at least one source of controlled ambient light within the housing for emitting ambient light having controlled spectral components visible to the user.

2. The apparatus as recited in claim 1 further including a plurality of structured light sources at spaced locations within the housing.

3. The apparatus as recited in claim 2 wherein the housing is generally rectangular shaped in plan view and the structured light sources are located at least proximate to each of the corners of the housing.

4. The apparatus as recited in claim 1 further including a plurality of light detectors at spaced locations within the housing.

5. The apparatus as recited in claim 4 wherein the housing is generally rectangular shaped in plan view and the light detectors are located at least proximate to each of the corners of the housing.

6. The apparatus as recited in claim 1 wherein the at least one light detector includes one or more optical components to pass predetermined spectral components of the structured light and to attenuate spectral components of the controlled ambient light.

7. The apparatus as recited in claim 6 wherein the one or more optical components include at least one of a band pass filter, a band block filter, a high pass filter and a low pass filter.

8. The apparatus as recited in claim 6 wherein the one or more optical components comprises a low pass filter that passes structured light at the red end of the spectrum and attenuates ambient light at the blue end of the spectrum.

9. The apparatus as recited in claim 6 wherein the one or more optical components comprises a high pass filter that passes structured light at the blue end of the spectrum and attenuates ambient light at the red end of the spectrum.

10. The apparatus as recited in claim 6 wherein the one or more optical components comprises a band pass filter that passes a narrow band of structured light and attenuates ambient light above and below the pass band.

11. The apparatus as recited in claim 6 wherein the at least one light detector further includes an optical imager for receiving light and converting the received light into electrical signals and an imager lens for focusing light onto the imager.

12. The apparatus as recited in claim 11 wherein the one or more optical components comprises at least one of one or more optical filters proximate to the imager lens and a coating on the imager lens.

13. The apparatus as recited in claim 11 wherein the one or more optical components comprises one or more optical filters fabricated of a polymeric material.

14. The apparatus as recited in claim 13 wherein the polymeric material is a polycarbonate.

15. The apparatus as recited in claim 11 wherein the optical imager comprises at least one of a VGA imager, an SVGA imager, an XVGA imager and a WXGA imager.

16. The apparatus as recited in claim 11 wherein the optical imager comprises at least one of a CMOS imager and a CCD imager.

17. The apparatus as recited in claim 11 wherein the optical imager comprises at least one of a black and white imager, a color imager and an IR imager.

18. The apparatus as recited in claim 11 wherein the optical imager is comprised of a VGA resolution black and white CMOS imager.

19. The apparatus as recited in claim 6 wherein the at least one source of controlled ambient light includes at least one of a fluorescent bulb, an incandescent bulb, a light emitting diode and a halogen bulb.

20. The apparatus as recited in claim 19 wherein the at least one source of controlled ambient light further includes at least one filter for filtering out broad spectral components of the emitted light.

21. The apparatus as recited in claim 20 wherein the spectral components that are filtered out are components below the blue end of the visible spectrum.

22. The apparatus as recited in claim 6 wherein the at least one structured light source projects structured light including the predetermined spectral components passed by the one or more optical components of the at least one light detector.

23. The apparatus as recited in claim 22 wherein the at least one structured light source includes at least one of a fluorescent bulb, an incandescent bulb, a halogen bulb, a light emitting diode, a laser and a laser diode.

24. The apparatus as recited in claim 6 wherein the at least one structured light source comprises at least one red laser having a wavelength of about 670 nm.

25. The apparatus as recited in claim 1 wherein the at least one structured light source projects structured light comprising at least one of a single point, multiple points, a single line, multiple lines, a predetermined pattern, a gradient and a diffraction image.

26. The apparatus as recited in claim 1 wherein the projected structured light is stationary, is moved in a predetermined path during scanning or changes in character, pattern or location.

27. A method for acquiring and processing data regarding physical attributes of a user in an accurate, repeatable and automated manner comprising the steps of:
providing a generally enclosed housing sized for receiving a user therein, the housing having an interior and an opening with a door for entry into and exit from the housing by the user, the door, when closed, preventing outside light from entering the housing;
projecting structured light onto at least one portion of the body of a user to be scanned within the housing for obtaining spatial measurements of the at least one portion of the body of the user;
detecting structured light reflected from the at least one portion of the body of the user and generating electrical signals corresponding to the spatial measurements of the at least one portion of the body of the user; and
emitting ambient light having controlled spectral components visible to the user during the scanning.
